(19) United States
(12) Patent Application Publication
(10) Pub. No.: US 2003/0219805 A1
(43) Pub. Date: Nov. 27, 2003

(54) Detection of alternative and aberrant mRNA splicing

(76) Inventors: Zvi Kelman, Gaithersburg, MD (US); Ralph Carmel, New York, NY (US)

Correspondence Address: INTELLECTUAL PROPERTY / TECHNOLOGY LAW, PO BOX 14329, RESEARCH TRIANGLE PARK, NC, 27709, US

(21) Appl. No.: 10/387,714

(22) Filed: Mar. 13, 2003

Related U.S. Application Data
(60) Provisional application No. 60/363,862, filed on Mar. 13, 2002.

(51) Int. Cl. C12Q 1/68; C12P 19/34

(52) U.S. Cl. 435/6; 435/91.2

(57) ABSTRACT

Disclosed is a method of determining alternatively spliced mRNAs by hybridization of mRNAs to exon/intron junction specific polynucleotide probes. Such polynucleotide probes allow the identification of alternative and aberrant splicing of known exons of a gene. These polynucleotide probes may by contained in DNA arrays to allow the screening of a large number of alternative and aberrant splicing variants.

### CROSS-REFERENCE TO RELATED APPLICATION

This application claims priority to U.S. Provisional Patent Application No. 60/363,862 filed on Mar. 13, 2002 in the names of Zvi Kelman and Ralph Carmel for “DETECTION OF ALTERNATIVE AND ABERRANT mRNA SPLICING.”

### BACKGROUND OF THE INVENTION

1. Field of Invention

The present invention relates to alternative splicing of pre-mRNA, and more particularly, to methods for determining different alternative splicing in mRNA and detecting aberrant splicing of pre-mRNA relative to diagnosing cellular alterations during disease development.

2. Description of the Related Art

Alternative splicing of pre-mRNA plays an important role in cell development and tissue-specific protein expression (for reviews see Lopez, A., 1998. Ann. Rev. Genet. 32: 279-305; Black, D., 2000. Cell 103: 367-370; Graveley, B., 2001. Trends Genet. 17:100-107). In addition, with the completion (or near completion) of the sequencing of the genome of several multicellular organisms, including humans and Drosophila, it has become apparent that alternative splicing plays a major role in protein diversity within an organism (Graveley, B., 2001. Trends Genet. 17:100-107). Further, in the last several years it has been shown that aberrant pre-mRNA splicing is also responsible for several human diseases, for example amyotropic lateral sclerosis (ALS) (Lin, C. et al., 1998. Neuron 20: 589-602), mytonic dystrophy (Philips, A. et al., 1998. Science 280:737-741), acute lymphoblastic leukemia (Goodman, P. et al., 2001. Oncogene. 20: 3969-3978), and rheumatoid arthritis (Shiozama, K. et al., 2001. Rheumatology. 40: 739-742). Efforts are being made to elucidate the mechanisms governing alternative splicing events. These and other studies will lead to the identification of more disease/disorders associated with alternative splicing.

The current approaches used to detect and diagnose cellular alterations during the development of disease rely either on mutational studies of the DNA (deletions, translocations and point mutations), alterations in RNA expression levels (Northern analysis and RT-PCR) or aberrant protein expression (ELISA, RIA and Western analysis). Although alterations in mRNA can be detected by techniques such as Northern blotting, such methods can detect only high copy number transcripts and require a large amount of the starting tissue or blood material. If the missing exon is small, methods such as Northern blotting are not useable. Northern analysis will not distinguish between several alternatively spliced products made from a single gene (Holmes, E. et al., 1992. Science 256:1205-1210; Graveley, B., 2001. Trends Genet. 17:100-107). Likewise, methods using ELISA and gel electrophoresis do not provide information regarding a specific exon inclusion or removal thereof because the gel may be able to determine that there are different size fragments indicating several alternative splicing forms of the gene are present but the gel will not be able to determine which exon has been spliced out of the different size fragments (Holmes, E. et al., 1992. Science 256:1205-1210).

Accordingly there is a need in the art for a method that identifies and differentiates between different spliced forms of mRNA and definitively determines the position and inclusion of specific exons in a mRNA.

### SUMMARY OF THE INVENTION

The present invention relates in one aspect to methods and tools to differentiate between different spliced forms of mRNA. In particular, the present invention provides for a method for detecting different alternative spliced forms of mRNA transcript from a gene of interest, the method comprising:

a) providing at least one sample polynucleotide comprising a nucleotide sequence representative of a mature mRNA transcript from a gene of interest;

b) generating at least one exon variable polynucleotide probe by combining at least a nucleotide sequence of the 3′ end of one exon and at least a nucleotide sequence of the 5′ end of another exon from the gene of interest; wherein the nucleotide sequences of the exons and intron-exon splice junctions were previously determined, and wherein the nucleotide sequence of the 3′ end of one exon and the 5′ end of another exon are combined in variable combinations of contiguous and/or noncontiguous exons;

c) exposing the at least one sample polynucleotide of step (a) to at least one exon variable polynucleotide probe under hybridizing conditions; and

d) detecting binding complexes between the at least one exon variable polynucleotide probe and the at least one sample polynucleotide of step (a).

The presence of a hybridization complex correlates with the presence of a sample polynucleotide containing adjoining exon sequences of a transcript of the gene of interest.

In another aspect, the present invention provides a method for determine alternative splicing of a mature mRNA transcript from a gene of interest, the method comprising:

a) contacting a sample polynucleotide comprising a nucleotide sequence complementary to the mature mRNA transcript with an exon variable polynucleotide probe under hybridizing conditions, wherein the exon variable polynucleotide probe comprises at least a 3′ end nucleotide sequence of one exon and a 5′ end nucleotide sequence of another exon from the gene of interest; wherein nucleotide sequences of at least the 3′ and 5′ end nucleotide sequences of the exons at a contiguous or noncontiguous intron-exon splice junction were previously determined, and wherein the nucleotide sequence of the 3′ end of one exon and the 5′ end of another exon are combined in variable combinations; and

b) detecting hybridized sample polynucleotides.

The present invention is used in identifying the expression patterns of different alternative spliced forms of mRNA (and thus the expression pattern of different proteins) during development and cell differentiation. It is therefore useful in identifying the expression patterns of the different proteins encoded by these alternatively spliced forms of mRNA.

The invention is useful as a diagnostic tool for disorders in which alternative splicing plays a role. As such, another aspect of the present invention relates to a diagnostic method for determining alternative splicing of a polynucleotide comprising the steps of

a) contacting a sample polynucleotide comprising a nucleotide sequence complementary to the mature mRNA transcript with an exon variable polynucleotide probe under hybridizing conditions, wherein the exon variable polynucleotide probe comprises at least a 3′ end nucleotide sequence of one exon and at least a 5′ end nucleotide sequence of another exon from the gene of interest; wherein nucleotide sequences of at least the intron-exon splice junctions were previously determined, and wherein the nucleotide sequence of the 3′ end of one exon and the 5′ end of another exon are combined in variable combinations of contiguous and/or noncontiguous exons; and

b) detecting hybridized sample polynucleotides; and

c) comparing the levels of the hybridized sample polynucleotide to the levels of a hybridized control polynucleotide, wherein the control polynucleotide hybridizes to a control polynucleotide exon probe comprising a 3′ exon junction sequence and a 5′ exon junction sequence from contiguous exons of a gene.

Polynucleotide probes comprising nucleotide sequences of a control sample or cell line may also serve as a control. Variations in the levels of hybridization complexes from the control probes indicates some aberrant or additional alternative splicing.

Other features and advantages of the invention will be apparent from the following detailed description, drawings and claims.

### BRIEF DESCRIPTION OF THE DRAWINGS

FIG. 1 illustrates the possible combinations of exons to be used as exon variable polynucleotide probes in the present invention.

FIG. 2 illustrates an exon variable polynucleotide probe (1/2) wherein the 5′ end of the polynucleotide probe is the 3′ exon junction sequence of exon 1 and the 3′ end of the polynucleotide probe is the 5′ exon junction sequence of exon 2.

### DETAILED DESCRIPTION OF PREFERRED EMBODIMENT

Disclosed is a method for determining alternative splicing of a mRNA transcript polynucleotide. The method identifies alternative splicing of a sample polynucleotide by its hybridization to exon variable polynucleotide probes. These probes are complimentary to both the 3′ exon junction sequence and the 5′ exon junction sequence of adjoining exon sequences of a gene transcript. Detection of the hybridized polynucleotide identifies a sample polynucleotide as containing two specific adjoining exon sequences of a gene transcript, with the understanding that the exons can be contiguous or non-contiguous.

In order to facilitate review of the various embodiments of the present invention and provide an understanding of the various elements and constituents used in making and using the present invention, the following terms used in the invention description have the following meanings.

The term “polynucleotide,” as used herein, is a composition or sequence comprising nucleotide subunits, wherein the subunits can be deoxyribonucleotides, ribonucleotides, deoxyribonucleotide analogs, ribonucleotide analogs or any combinations thereof. Deoxyribonucleotide analogs and ribonucleotide analogs are defined as deoxy- or ribonucleotides that are not found in nature. Those nucleotides found in nature are thymidine, deoxycytidine, deoxyguanidine, deoxyadenine, deoxyuridine, cytidine, guanidine, adenine and uridine. Analogs of deoxyribonucleotides and ribonucleotides are well known in the art and include, but are not limited to analogs such as cytidine, guanidine, adenine, uridine, thymidine, deoxycytidine, deoxyguanidine, deoxyadenine and deoxyuridine labeled with a fluorescent compound, radioisotope, antibody-recognized antigen, biotin or digoxigenin. Analogs also include but are not limited to bromodeoxyuridine, bromouridine, methyl uridine, hydroxymethyl uridine, fluorodeoxyuridine, fluorouridine, chlorodeoxyuridine, chlorouridine, iododeoxyuridine, jodouridine, chlorodeoxyuridine, amino allyl deoxyuridine, amino allyl uridine, thio uridine, ethenodeoxyadenosine, benzoyl deoxyadenosine, deazadeoxyguanosine, methyldeoxycytidine and methyldeoxyguanosine.

The term “exon,” as used herein, is a sequence of nucleotides from a gene, which encodes a protein or portions of a protein. An intron or intervening sequence is defined as a sequence of nucleotides, which interrupt the protein-coding sequence of a gene. Introns are transcribed into the pre-RNA but are cut out so that they are not translated into protein.

The term “exon splice junction sequence” or exon junction sequence, as used herein, is a sequence of nucleotides in the exon immediately adjacent to an intron sequence. In a gene, these junction sequences are located at the 3′ and 5′ end of the exon and immediately adjacent to an intron sequence.

The term “contiguous exons,” as used herein, are exons of a gene which are separated by an intron in the gene and which are adjacent to each other on a mRNA transcript of the gene. “Noncontiguous exons” are exons of a gene that are separated by more than one intron or are separated by at least one other exon.

The term “exon variable polynucleotide probe,” as used herein, is a probe used for detecting polynucleotides that have a similar or identical sequence that hybridize to the probe. The probe can be labeled and may form a hybridization complex with a sample polynucleotide due to the complementarity of a nucleotide sequence in the probe with a nucleotide sequence in the sample polynucleotide.

The term “microarray,” as used herein, refers to an array of distinct polynucleotides or oligonucleotides arranged on a substrate, such as paper, nylon or other type of membrane, filter, chip, glass slide, or any other suitable solid support.

The term “hybridization,” as used herein, is the process of two complimentary strands of two polynucleotides forming a double stranded molecule as a result of base-pairing between the individual nucleotides of the two polynucleotides. The two strands of polynucleotides may be completely complimentary and defined as two strands of polynucleotides having no corresponding mismatched nucleotide base pairs to the extent of the shortest polynucleotide strand. Two strands of polynucleotides may be partially complimentary and defined as two strands of polynucleotides having both corresponding matched and mismatched nucleotide base pairs.

The term “hybridization complex,” as used herein, refers to a complex formed between two nucleic acid sequences by virtue of the formation of hydrogen bonds between complementary bases. A hybridization complex may be formed in solution or between a sample polynucleotide sequence present in solution and an exon variable polynucleotide probe immobilized on a solid support (e.g., paper, membranes, filters, chips, pins or glass slides, or any other appropriate substrate to which cells or their nucleic acids have been fixed).

The term “stringent conditions,” as used herein, refers to conditions that permit hybridization between a sample polynucleotide sequence and an exon variable polynucleotide probe sequence. Suitably stringent conditions can be defined by, for example, the concentrations of salt or formamide in the prehybridization and hybridization solutions or by the hybridization temperature, and are well known in the art. In particular, stringency can be increased by reducing the concentration of salt, increasing the concentration of formamide or raising the hybridization temperature.

For applications requiring a high degree of selectivity, relatively stringent conditions are employed to form a hybridization complex. For example, relatively low salt and/or high temperature conditions, such as provided by a NaCl salt concentration in the range from about 0.02M to about 15M and at temperatures in the range from about 50° C. to about 70° C., will be selected. Those conditions are particularly selective, and tolerate little, if any, mismatch between the probe and the template or target strand. Effective high stringency hybridization conditions may include: 6×SSPE, S×Denhardt's reagent, 50% formamide, 42° C., 0.5% SDS, 100 ug/ml sonicated denatured calf thymus or salmon sperm DNA.

Medium stringency hybridization conditions may include the following conditions: 6×SSPE, 5×Denhardt's reagent, 42° C., 0.5% SDS, 100 ug /ml sonicated denatured calf thymus or salmon sperm DNA; and low stringency hybridization conditions may include the following conditions: 6×SSPE, S×Denhardt's reagent, 30° C., 0.5% SDS, 100 ug/ml sonicated denatured calf thymus or salmon sperm DNA.

Formulae for buffers that be used for hybridizations in the present invention include: 20×SSPE: 3.6 M NaCl, 0.2 M phosphate, pH 7.0, 20 mM EDTA. 50×Denhardt's reagent: 5 g FICOLL Type 400, 5 g polyvinylpyrrolidone, 5 g bovine serum albumin and water to 500 ml.

The term “isolated polynucleotide,” as used herein, is a polynucleotide, which is considerably free from naturally occurring cellular components. An isolated polynucleotide would also include the polynucleotide enriched in concentration over its concentration in the cell. Any amplified polynucleotide is considered to be free from cellular components.

The exon variable polynucleotide probes may be positioned on a substrate suitable for adhering such polynucleotide probes. Suitable substrates may include any surface, which binds polynucleotides including, but are not limited to, nitrocellulose, nylon, glass, polyvinylidene fluoride and polystyrene. Substrates may also be treated with compounds to improve polynucleotide binding, such as poly-L-lysine on glass. Polynucleotides may be covalently attached to a substrate, with methods of covalent attachment to a solid support well known in the art (Zammatteo, N. et al., 1996. Anal. Biochem. Vol.236, pp. 85-94).

The sample polynucleotides and probes used in the present invention may be labeled which allows detection of that polynucleotide. Suitable detectable labels may include, but are not limited to, radioisotopes, biotinylated compounds, digoxigenin compounds, fluorophores, antibody-recognized antigens and enzymes. Examples of fluorophores include fluorescein, rhodamine, CY3 and CY5. Biotinylated compounds include biotin-labeled nucleotides and biotin attached to a tinker with an amino-reactive group. The amino-reactive group is able to form a covalent bond with nucleotide bases comprising an amino group. Digoxigenin compounds include digoxigenin-labeled nucleotides and digoxigenin attached to a linker with an amino-reactive group. Antibody-recognized antigens are antigens that antibodies bind. The bound antibodies are then visualized by methods well known in the art. Antigens that are detectable by an antibody include bromodeoxyuridine and methylated bases of nucleotides. Examples of enzymes include horseradish peroxidase and alkaline phosphatase. These enzymes are able to catalyze various detectable substrates.

The examples of detectable labels listed above are merely meant to be illustrative and are by no means meant to be a limiting or exhaustive list. Methods of labeling DNA or RNA with a detectable label are well known in the art and include nick translation, RNA transcription, PCR amplification in the presence of a labeled nucleotide or direct chemical reaction of a label with reactive groups on the polynucleotide.

Analysis of alternative splicing of pre-mRNA begins with the isolation of mRNA from the desired tissue, blood or tissue culture cells. The isolated mRNA may be amplified to increase the amount usable as a sample mRNA by amplifying techniques such as a RT-PCR reaction with primers specific to the nucleotide sequence to be studied. Total cellular RNA can be extracted from tissues or cells by any of several well known techniques and used as a template for RT. Generally, the RNA is primed using random primers. The technique uses the ability of reverse transcriptase (RT) to convert RNA into single-stranded cDNA. The cDNA, which is produced during the RT reaction represents a picture of the pattern of genes that are expressed at the time RNA was extracted from the sample. A small amount of the cDNA, used as a template, is then added to a PCR reaction containing primers specific or degenerate to the sequences that are to be amplified. The design of the primers is dependent upon the sequence of the DNA or RNA that is desired to be analyzed. The PCR technique is carried out through many cycles (20-50) of melting the cDNA template at high temperature, such as a denaturing temperature, and then cooling to a temperature that allows the primers to anneal to the complimentary sequences through the cooling process and then replicating the template with DNA polymerase. Preferably, the RT-PCR reaction amplifies the entire mRNA transcript (namely using primers with the most 3′ and 5′ end sequences).

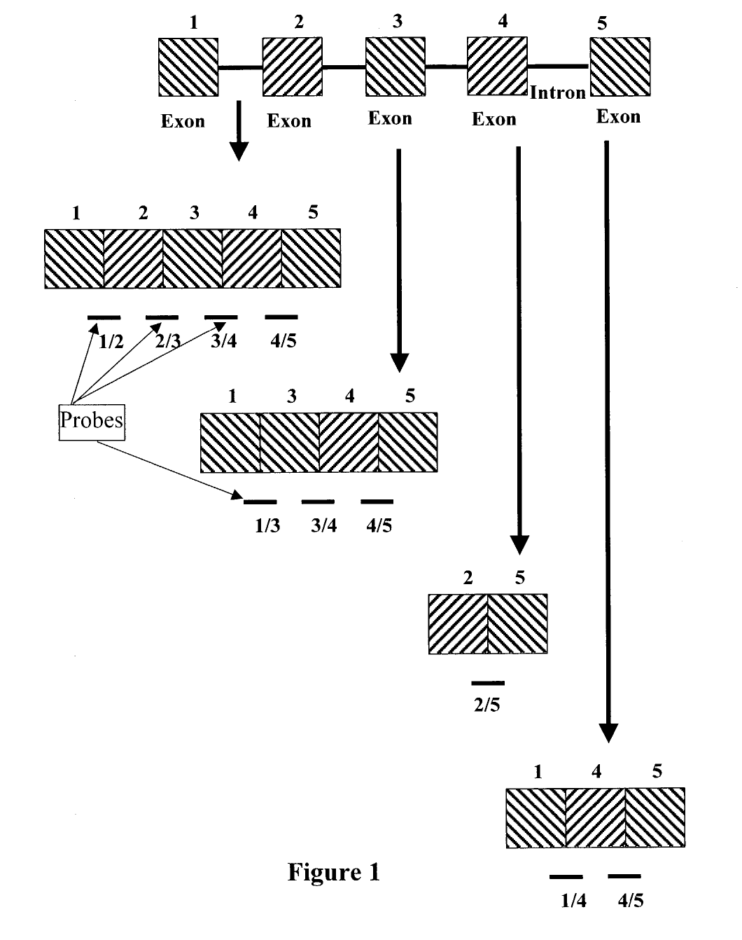

The RT-PCR may be performed by adding radioactive dNTPs to the PCR reaction mixture to incorporate labels into the amplified transcripts. Alternatively, labeled primers may be used. Several type of labeling can be used (for example, radioactive or fluorescent labels), depending on the technology used. The products of RT-PCR may be analyzed by separation in agarose gels, followed by ethidium bromide staining and visualization with UV transillumination. The RT-PCR transcript products can then be used for hybridization to exon variable polynucleotide probes of the present invention encompassing different exon splice site regions of the mature mRNA (see FIG. 1).

Before the exon variable polynucleotide probes can be generated, the DNA sequence of the gene of interest must be determined, and specifically the nucleotide sequences included in each exon and particularly the nucleotide sequences in the exons that are adjacent to each exon-intron splice junction. Sequencing of DNA can be accomplished by either chemical or enzymatic means that are well known to those skilled in the art. Chemical techniques for sequencing rely on the nucleotide specific chemical cleavage of DNA, such as the Maxam and Gilbert method. Enzymatic sequencing, such as Sanger sequencing method may also be used. Both methods are well known to those skilled in the art.

The nucleotide sequences of the different exons at the exon-intron splice junctions must be known to perform the present invention. During the processing of a primary RNA transcript, introns are spliced from a mRNA transcript and the some or all of the remaining exons are connected into a continuous mRNA sequence. The splicing signal in the primary transcript has three major features, including a 100% conserved GU at the 5′ end of the intron; a 100% conserved AG at the 3′ end of the intron; and an A nucleotide about 20-30 nucleotides from the 3′end of the intron that is called a branch point and is 100% conserved. Thus, these splicing signals may be used as a roadmap to easily determine the exon sequences adjacent to the introns splice sites in the gene of interest. With the conserved and available signals, the exon sequences and specifically the exon-intron splice sites can be identified by using computer programs available on the commercial market and well known to those skilled in the art. Specifically, “Genscan ™” (Stamford University, CA) allows a researcher to easily discern intron/exon splice sites. Once the genomic DNA sequence is determined, the sequence can be simply pasted into the Genscan™ and the program scans the sequence for the splicing signals, as discussed above. An output table of predicted exons will be generated along with the specific sequences of such exons.

Once the sequence of the gene that codes for the pre-mRNA is determined, the exon variable polynucleotide probes of the present invention may be prepared. The exon variable polynucleotide probes can be prepared by any method well known in the art for the synthesis of DNA and RNA molecules. For example, nucleic acids may be chemically synthesized using commercially available reagents and synthesizers (see e.g. Gail, 1985, Oligonucleotide Synthesis: A Practical Approach, IRL Press, Oxford England). Alternatively, RNA molecules can be generated by in vitro and in vivo transcription of DNA sequences encoding the RNA molecules. Such sequences can be incorporated into a wide variety of vectors that incorporate suitable RNA polymerase promoters such as T7 or SP6 polymerase promoters. RNA may be produced in high yield via in vitro transcription using plasmids such as SPS65 (Promega Corporation, Madison, Wis.).

As this invention is a diagnostic tool, the intron/exon borders and the alternatively spliced exon(s) must be known. Thus, the polynucleotides used as probes must encompass at least the 3′ end or 5′ end of alternatively spliced exons. For example, if the nucleotide sequence of exon three (3) is missing from an mRNA transcript which would normally comprise the nucleotide sequence of exons 1,2, 3 and 4, a probe polynucleotide comprising a DNA sequence complimentary to the 3′ end of exon two (2) and the 5′ end of exon four (4) would be expected to hybridize to the mRNA transcript missing the nucleotide sequence of exon three (3).

FIG. 1 illustrates the exon variable polynucleotide probes that hybridize to the sample polynucleotide sequences. The hatched boxes represent exon sequences 1 through 5 that are subsequently transcribed and spliced. The short black lines underneath the exons are specific polynucleotide probes that are complimentary to the corresponding transcript nucleotide sequences corresponding to the specific exon-exon junctions. These specific polynucleotide probes are combined into exon variable polynucleotide probes that can be stabilized on a substrate for hybridizing with complementary sequences in mRNAs. A sufficient amount of probes should be constructed to include random and variable combinations of all exons determined to be included in the gene of interest or mRNA.

Depending on how the gene comprising the five exons is transcribed and spliced, several transcripts of the gene are possible. The probes, in FIG. 1, are identified by the adjoining exons, e.g. the probe identified as 1/3 represents a polynucleotide probe comprising a nucleotide sequence, which is complimentary to the 3′ nucleotide sequence of exon 1 and to the adjoining 5′ nucleotide sequence of exon 3. Accordingly, the probe identified as 2/5 represents a polynucleotide probe comprising a nucleotide sequence which is complimentary to the 3′ nucleotide sequence of exon 2 and to the adjoining 5′ nucleotide sequence of exon 5.

In an alternative approach, the RT-PCR step is omitted and the mRNA is labeled directly (Lockhart, D. and Winzeler, E., 2000. Nature 405: 827-836), followed by hybridization of the mRNA to the exon variable polynucleotide probes. In yet another approach, mRNA is amplified as antisense RNA (Van Gelder, R. et al. 1990. Proc. Natl. Acad. Sci. USA. Vol. 87, pp 1663-1 667) which is then hybridized to the exon variable polynucleotide probes.

As the present invention detects only spliced RNA products, the results are not rendered ambiguous if total RNA is included, and as such, there is no requirement for mRNA purification. Furthermore, contamination by genomic DNA will not affect the results and thus makes the material preparation relatively easy. When this method is used in conjunction with chip analysis and differently labeled probes, several genes can be analyzed simultaneously. Furthermore, the invention will enable large-scale base screening.

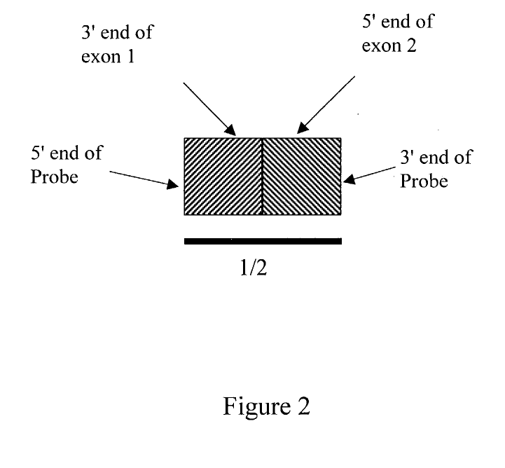

A preferred embodiment of the invention is a method for determining alternative splicing of a sample polynucleotide comprising hybridizing the sample polynucleotide to an exon variable polynucleotide probe comprising a 3′ exon junction sequence and 5′ exon junction sequence, wherein the 5′ end of the polynucleotide probe is the 3′ exon junction sequence of one exon and the 3′ end of the polynucleotide probe is the 5′ exon junction sequence of another exon, as shown in FIG. 2.

In another embodiment of the invention, the 3′ exon junction sequence and 5′ exon junction sequence are from contiguous exons of the gene.

In yet a further embodiment of the invention, the 3′ exon junction sequence and 5′ exon junction sequence are from noncontiguous exons of the gene.

Generally, the exon variable polynucleotide probe length is a sufficient length of nucleotides to provide a complementary binding site for preferential binding of mRNA having a complementary exon sequence. Preferably, the probes have about 20 to about 200 nucleotides, and more preferably from about 41-60 nucleotides, and most preferably from about 18 to about 20 nucleotides.

An exon variable polynucleotide probe of the present invention preferably comprises an equivalent number of nucleotides from each contiguous or non-contiguous exon. An equivalent number of nucleotides means that the number of nucleotides from each combined differs by no more than 5 nucleotides, more preferably differs by no more than 3 nucleotides, and most preferably, the number of nucleotides is exactly the same. Thus, a preferred probe may be 20 nucleotides wherein 10 nucleotides of the 3′ end of one exon and 10 nucleotides of the 5′ end of another exon, whether contiguous, or noncontiguous are fused into a single 20 nucleotide probe.

The exon variable polynucleotide probes and determining the nucleotide sequences of the RNA transcripts are produced using procedures well known in the art. Examples include: polymerase chain reaction (PCR; Sambrook, et al., Molecular cloning; A laboratory Manual: Vol. 1-3, Cold Spring Harbor Laboratory Press, Cold Spring Harbor, N.Y. (1989)); DNA synthesis using an Applied Biosystems DNA synthesizer (Perkin Elmer ABI 3948, using the standard cycle as according to procedures provided by the manufacturer); agarose gel electrophoresis (Ausubel, Brent, Kingston, Moore, Seidman, Smith and Struhl, Current Protocols in Molecular Biology: Vol. 1 and 2, Greene Publishing Associates and Wiley-Interscience, New York (1990); (See also Current Protocols in Immunology); analysis of tissue culture supernatants and cell lysates by sodium dodecylsufate-polyacrylamide gel electrophoresis (SDS-PAGE; Harlow and Lane. Using Antibodies, A Laboratory Manual, Cold Spring Harbor Laboratory Press, NY, (1988)) and immunoblotting (Harlow and Lane); and quantitative reverse transcriptase (RT)-PCR (Ausubel, et al., IN: Current Protocols in Molecular Biology: Vol. 1 and 2, Greene Publishing Associates and Wiley-Interscience, New York (1990)), using the Thermoscript RT-PCR System (Life Technologies, Gaithersburg Md.; cat #11146-016). All references are hereby incorporated herein by reference for all purposes.

We claim:

1. A method for detecting different alternative spliced forms of a mRNA transcript from a gene of interest, the method comprising:
a) providing at least one sample polynucleotide comprising a nucleotide sequence representative of the mRNA transcript;
b) generating at least one exon variable polynucleotide probe by combining at least a nucleotide sequence of a 3′ end of one exon and at least a nucleotide sequence of a 5′ end of another exon from the gene of interest; wherein the nucleotide sequences of the exons and intron-exon junctions were previously determined, and wherein the nucleotide sequence of the 3′ end of one exon and the 5′ end of another exon are combined in variable combinations of contiguous and/or noncontiguous exons;
c) exposing the at least one sample polynucleotide of step (a) to at least one exon variable polynucleotide probe under hybridizing conditions; and
d) detecting binding complexes between the at least one exon variable polynucleotide probe and the at least one sample polynucleotide of step (a), wherein the presence of a hybridization complex correlates with the presence of a sample polynucleotide containing adjoining exon sequences of a transcript of the gene of interest.

2. The method according to claim 1, wherein the at least one exon variable polynucleotide probe is stabilized on a surface.

3. The method according to claim 1, wherein the 5′ end of the variable exon polynucleotide probe comprises a 3′ exon junction sequence and the 3′ end of the variable exon polynucleotide probe comprises a 5′ exon junction sequence.

4. The method according to claim 3, wherein the 3′ exon junction sequence and 5′ exon junction sequence are from contiguous exons of the gene of interest.

5. The method according to claim 3, wherein the 3′ exon junction sequence and 5′ exon junction sequence are from noncontiguous exons of the gene of interest.

6. The method according to claim 1, wherein the hybridizing conditions comprise high stringency conditions at temperatures in the range from about 50° C. to about 70° C.

7. The method according to claim 1, wherein the variable exon polynucleotide probe comprises a sufficient number of nucleotides to hybridize a polynucleotide sequence comprising a complementary sequence to the exon variable polynucleotide probe.

8. The method according to claim 1, wherein the variable exon polynucleotide probe comprises 20-60 nucleotides in length.

9. The method according to claim 1, wherein the polynucleotide probe is 18-20 nucleotides in length.

10. The method according to claim 1, wherein the sample polynucleotide has been amplified.

11. The method according to claim 1, wherein the sample polynucleotide is labeled with a detectable label.

12. The method according to claim 11, wherein the detectable label is selected from the group consisting of radioisotopes, biotinylated compounds, digoxigenin compounds, fluorophores, antibody-recognized antigens and enzymes.

13. A method for determine alternative splicing of a mature mRNA transcript from a gene of interest, the method comprising:
a) contacting a sample polynucleotide comprising a nucleotide sequence complementary to the mature mRNA transcript with a variable exon polynucleotide probe under hybridizing conditions, wherein the variable exon polynucleotide probe comprises at least a 3′ end nucleotide sequence of one exon and a 5′ end nucleotide sequence of another exon from the gene of interest; wherein nucleotide sequences of at least the 3′ and 5′ end nucleotide sequences of the exons at a contiguous or noncontiguous intron-exon splice junction were previously determined, and wherein the nucleotide sequence of the 3′ end of one exon and the 5′ end of another exon are combined in variable combinations; and
b) detecting hybridized sample polynucleotides.

14. The method according to claim 13, wherein the hybridizing conditions comprise high stringency hybridization conditions.

15. The method according to claim 13, wherein the at least one exon variable polynucleotide probe is stabilized on a surface.

16. The method according to claim 13, wherein the 5′ end of the variable exon polynucleotide probe comprises a 3′ exon junction sequence and the 3′ end of the variable exon polynucleotide probe comprises a 5′ exon junction sequence.

17. The method according to claim 16, wherein the 3′ exon junction sequence and 5′ exon junction sequence are from contiguous exons of the gene of interest.

18. The method according to claim 16, wherein the 3′ exon junction sequence and 5′ exon junction sequence are from noncontiguous exons of the gene of interest.

19. The method according to claim 13, wherein the exon variable polynucleotide probe comprises an equal number of nucleotides from the 3′ end of one exon and the 5′ end of another exon.

20. The method according to claim 13, wherein the at least one exon variable polynucleotide probe is in solution.

21. A diagnostic method for determining alternative splicing of a polynucleotide comprising the steps of
a) contacting a sample polynucleotide comprising a nucleotide sequence complementary to the mature mRNA transcript with an exon variable polynucleotide probe under hybridizing conditions, wherein the exon variable polynucleotide probe comprises at least a 3′ end nucleotide sequence of one exon and a 5′ end nucleotide sequence of another exon from the gene of interest; wherein nucleotide sequences of at least the intron-exon junctions were previously determined, and wherein the nucleotide sequence of the 3′ end of one exon and the 5′ end of another exon are combined in variable combinations of contiguous and/or noncontiguous exons; and
b) detecting hybridized sample polynucleotides; and
c) comparing the levels of the hybridized sample polynucleotides to the levels of a hybridized control polynucleotide, wherein the control polynucleotide hybridizes to a control polynucleotide probe comprising a 3′ exon junction sequence and a 5′ exon junction sequence from contiguous exons of a gene.

22. The method according to claim 21, wherein the at least one exon variable polynucleotide probe is stabilized on a surface.

23. The method according to claim 21, wherein the 5′ end of the variable exon polynucleotide probe comprises a 3′ exon junction sequence and the 3′ end of the exon variable polynucleotide probe comprises a 5′ exon junction sequence.

24. The method according to claim 23, wherein the 3′ exon junction sequence and 5′ exon junction sequence are from contiguous exons of the gene of interest.

25. The method according to claim 23, wherein the 3′ exon junction sequence and 5′ exon junction sequence are from noncontiguous exons of the gene of interest.

26. The method according to claim 21, wherein the hybridizing conditions comprise high stringency conditions at temperatures in the range from about 50° C. to about 70° C.

27. The method according to claim 21, wherein the exon variable polynucleotide probe comprises a sufficient number of nucleotides to hybridize a polynucleotide sequence comprising a complementary sequence to exon variable polynucleotide probe.

28. The method according to claim 21, wherein the exon variable polynucleotide probe comprises 20-60 nucleotides in length.

29. The method according to claim 21, wherein the exon variable polynucleotide probe is 18-20 nucleotides in length.

30. A method for determining alternative splicing of a mRNA transcript polynucleotide from a gene of interest, the method comprising identifying alternative splicing of a sample polynucleotide representative of the mRNA transcript, by hybridization thereof to at least one exon variable polynucleotide probe comprising a 3′ exon junction sequence and a 5′ exon junction sequence of adjoining exon sequences of a transcript of the gene of interest, to detect a sample polynucleotide containing two specific adjoining exon sequences of the transcript of the gene of interest.
